(19) United States
(12) Patent Application Publication
(10) Pub. No.: US 2022/0355124 A1
(43) Pub. Date: Nov. 10, 2022

(54) METHOD AND APPARATUS FOR DIAGNOSIS AND TREATMENT OF CARDIAC CONDUCTION ABNORMALITIES

(71) Applicant: WEINBERG MEDICAL PHYSICS INC., North Bethesda, MD (US)

(72) Inventors: Irving WEINBERG, North Bethesda, MD (US); Oleg UDALOV, North Potomac, MD (US)

(21) Appl. No.: 17/738,770

(22) Filed: May 6, 2022

Related U.S. Application Data
(60) Provisional application No. 63/184,940, filed on May 6, 2021.

(51) Int. Cl. A61N 1/40

(52) U.S. Cl. CPC A61N 1/40

(57) ABSTRACT

Disclosed apparatuses and methods apply electric fields deep in a body by selectively actuating multiple magnetic modules about the body sequentially in time. The electric field induced in regions of the body from such actuations may have a different frequency, depending on the depth of the region.

### CROSS REFERENCE AND PRIORITY CLAIM

This application claims priority to U.S. Provisional Patent Application Ser. No. 63/184,940, entitled “METHOD AND APPARATUS FOR DIAGNOSIS AND TREATMENT OF CARDIAC CONDUCTION ABNORMALITIES,” filed 6 May 2021, the entirety of which is incorporated by reference.

### FIELD

Disclosed embodiments are drawn to methods and apparatuses for diagnosing and treating cardiac conduction abnormalities in animals, including humans. In particular, disclosed embodiments may be used in medical and veterinary applications.

Presently cardiac conduction abnormalities are treated with broadly applied electrical currents (for example, cardioversion for atrial fibrillation) or with invasive procedures that stimulate, stun, or ablate portions of the heart or other organs.

### SUMMARY

Disclosed apparatuses and methods are provided that apply electric fields deep in a body by selectively actuating multiple magnetic modules about the body sequentially in time. The electric field induced in regions of the body from such actuations may have a different frequency, depending on the depth of the region. Since the likelihood of nerve modulation is dependent on the rate of change (i.e., frequency) of the electric field, nerves in different regions may be differentially modulated. This principle can be used to apply cardiac stimulation, temporary inhibition, or permanent ablation of nerves and conduction pathways deep in the heart or other nerves.

In some embodiments, the apparatuses and methods further include collecting an image of the body part using at least one of the magnetic modules. In some embodiments, the magnetic modules includes a magnetizable material and a coil of electrically conductive material. In some embodiments, the electric fields induced in the body part are high enough to cause temporary or permanent changes in electrical conduction or conductivity. In some embodiments, sensors are provided that are configured to detect electrical or magnetic activity in the body part.

### BRIEF DESCRIPTION OF THE FIGURES

Aspects and features of the disclosed embodiments are described in connection with various figures, in which:

FIG. 1 shows an assembly of modules near to or surrounding a body part;

FIG. 2 shows two actuation configurations of assembly near to or partially surrounding body part and graphs of one component of vector potential spatial distribution in a certain slice of the body corresponding to each respective actuation configurations according to the disclosed embodiments;

FIGS. 3a-d illustrates how selective actuation of modules in the assembly of disclosed embodiments may rotate the vector potential in space as a function of time, with activated magnets for several time steps are shown with darker shading in FIG. 3a, the absolute value of the vector potential spatial distribution is shown in FIG. 3b, an illustration of the electric field induced in the body part as a function of time for two locations is graphed in FIG. 3c and the two locations are shown in FIG. 3d;

FIG. 4 illustrates how the regions of electrical field may be changed in time through changes in module activation so as to yield a smaller region of electrical change in tissue in accordance with the disclosed embodiments;

FIG. 5 is a flow chart illustrating an embodiment of the method by applying configurations as in FIGS. 2-4, sometimes at the same time;

FIG. 6 is a flow chart illustrating an embodiment of the method in which a magnetic resonance image is used to examine whether a region and regimen is appropriate for application of electrical fields before the electrical fields have been applied according to the disclosed embodiments; and

FIG. 7 is a block diagram of a control system and power source according to the disclosed embodiments.

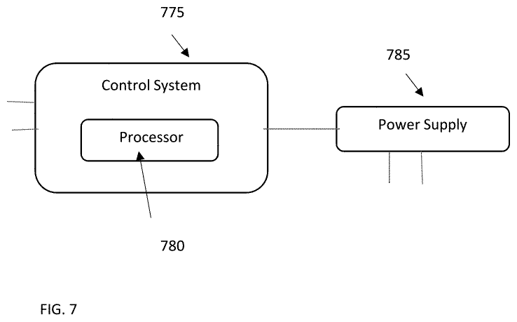

### DETAILED DESCRIPTION

The present invention will now be described in connection with one or more embodiments. It is intended for the embodiments to be representative of the invention and not limiting of the scope of the invention. The invention is intended to encompass equivalents and variations, as should be appreciated by those skilled in the art.

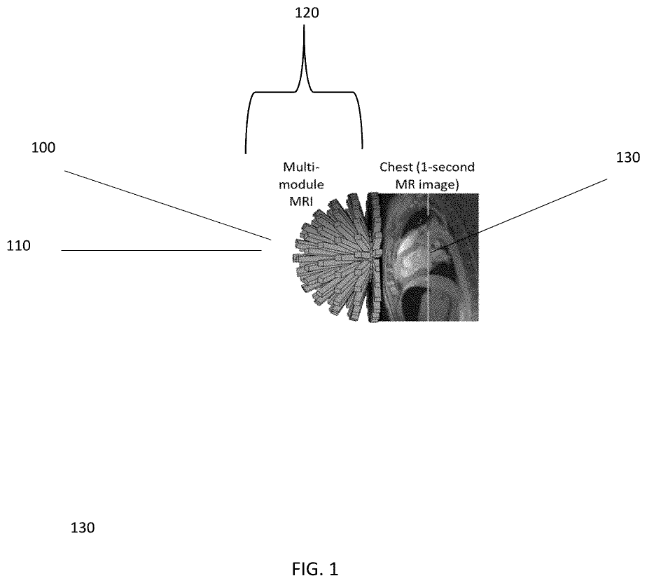

As shown in FIG. 1, a device is provided that consists of an assembly 120 of at least two magnetic modules 100 and 110, each the module containing magnetizable material or another source of magnetic fields. The assembly is shown near to or surrounding a body part 130, which may be a heart or other tissue containing nerves. It should be understood that in FIG. 1, magnetic modules 100 and 110 are shown as examples of modules, and they may be of different shapes, sizes, or configurations. In an embodiment, each magnetic module may constitute an electropermanent magnet, containing a core of AlNiCo or other magnetizable rods or other shapes surrounded in part by one or more coils of electrically conductive material. The magnetic module may be actuated by causing electrical currents to flow in the one or more coils. The magnetic module may have one or more magnetic sensors for adjustment or monitoring of the magnetic field created by the magnetic module or other magnetic modules in the assembly, or to detect magnetic activity in the body part (for example, to obtain a gated image that is binned or otherwise corrected for the phase of the cardiac or respiratory cycle).

For the purposes of this description, the terms “modules” and “magnetic modules” are used interchangeably. In an embodiment, the magnetic module may contain coils without a core to create a magnetic field. One or more magnetic modules in the assembly may be actuated (“fired’) at a different time than other magnetic modules in the assembly. The assembly may surround or be near a body part 130. By “near”, it is meant less than 100 cm in distance. The body part may be a heart, tooth, joint, or other structure containing nerves or neurons or other tissue that may be physiologically affected by magnetic or electrical fields. Such physiological effect may include modulation, stimulation, inhibition, transmission, or blocked transmission of nerve impulses. In the case of the teeth, the effect may be anesthetic, thereby reducing or eliminating the need for drug anesthesia. The use of very fast MRI pulse sequences may be advantageous for studies involving the teeth, since the decay time of solid-associated protons is quick. The effect may be temporary (for use in mapping or treatment planning prior to therapy, or as anesthesia) or permanent (as for therapy). The magnetic module may include a coil or other magnetic or electrical sensor for transmission or reception of radiofrequency or other electromagnetic energy from a body part. Alternatively, the coil or sensor may be separate from the module.

For the purposes of this description, the terms “neuron”, “nerve” and “neuronal tissue” are used interchangeably to mean tissue containing neurons or nerves or tissues stimulated or otherwise affected by neurons or nerves (for example, muscles or muscle groups or other electrically conductive tissues in the heart, gut, inner ear, optic nerve, nerve root or other locations). The term “stimulation” is intended to mean increasing the firing frequency, but is also used to mean any modulation, for example, interference with transmission or inhibition of impulses conveyed by nerves or neurons. The term “actuation” is used interchangeable with “activation” and means the changing of the magnetic state of one or more magnetic modules.

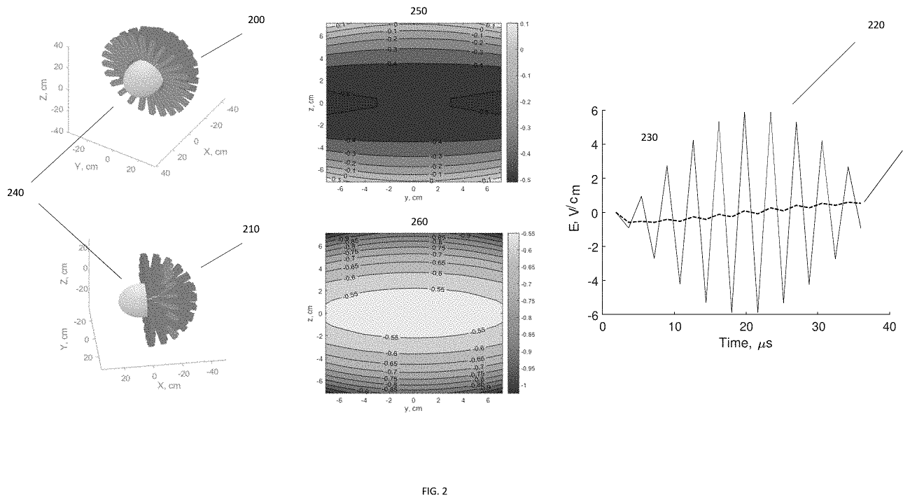

FIG. 2 shows two configurations of assembly 120 near to or partially surrounding body part 240. In actuation configuration 200, a set of modules is magnetized differentially with respect to the rest of the assembly. In actuation configuration 210, a different set of modules is magnetized with respect to the rest of the assembly. Graph 250 and 260 shows one component of vector potential spatial distribution in a certain slice of the body 240, the distribution corresponding to actuation configurations 200 and 210, respectively. The graph (220) shown illustrates the electric field as a function of time near the surface of the body part and deep in the body part 230 as the magnetic configurations are changed from 200 to 210 as a function of time.

FIG. 2 shows that as one or more sets of magnetic modules fire, examples of sets being 200 and 210, electrical fields are induced in the body part 240. The different sets of magnetic modules fired in 200 and 210 create electrical fields in the shallow portion of the body part that vary as the modules are fired, as shown in trace 220. Deep in the body part, the electrical fields change more slowly in time, as shown in trace 230. Such a difference in the timescale of electric field application in the superficial and deeps area may be achieved through a chosen set of actuated magnets and chosen extent of actuation for each magnet. An example of vector potential spatial distribution (one component only) is shown in FIG. 2 at 250 and 260. Two actuation configurations may be chosen in such a way that in both of them the vector potential in the deep region is similar, while the vector potential at the superficial region is very different. Therefore, in some embodiments, an electric field quickly oscillates at the surface and slowly varies in the middle by slowly increasing and decreasing the actuation strength.

An important aspect of the disclosed embodiments is that they exploit a physiological phenomenon, in which the effect of changing electrical fields applied to neuronal or nervous tissue is highly dependent on the duration of the change. This phenomenon was described by Weinberg et al in the publication entitled “Increasing the oscillation frequency of strong magnetic fields above 101 kHz significantly raises peripheral nerve excitation thresholds.,” published in Medical physics, vol. 39, no. 5, pp. 2578-83, May 2012, doi: 10.1118/1.3702775. As discussed in that publication, nerves have a much higher threshold for stimulation when magnetic fields with rise or fall times of less than 10 microseconds (or with frequencies greater than 101 kHz) are applied to the nerves, as compared to magnetic fields that rise or fall more slowly. Thus the disclosed embodiments permit the selective modulation of deep structures. It should be understood that modulation or stimulation may not be “all or nothing” states, and so the selective modulation may be the modulation to a lesser or greater extent. For purposes of this description, the term “frequency” is used interchangeably with rise and fall time, whereby a high frequency means that the rise and/or fall time is short, and a low frequency means that the rise and/or fall time is long. For example, a 100 kHz frequency pulse sequence implies that the rise time is about 10 microseconds and the fall time is about 10 microseconds.

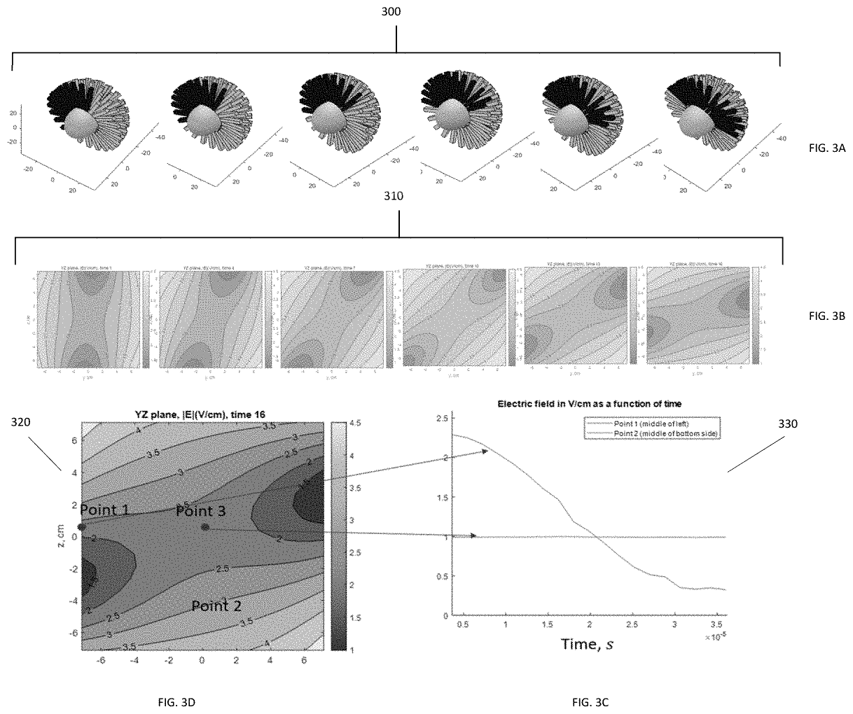

FIGS. 3a-d illustrate embodiments in which the location and extent of the electric field modulation region is established. Particularly, FIG. 3 illustrates how selective actuation of modules in the assembly may rotate the vector potential, and, therefore, also rotate the magnetic and electric fields in space as a function of time. Activated magnets for several points in time are shown 300 with darker shading. The activated sector of magnetic modules rotates at each next operation. By successively selecting different sets of modules for magnetization 300, the vector potential (and, therefore, magnetic and electric fields) 310 can rotate in the body part. The absolute value of the vector potential spatial distribution 310 is shown in FIG. 3b. An illustration of the electric field induced in the body part as a function of time for two locations (one deep in the body part and one superficially located in the body part) is graphed in 330. The locations are shown in 320. The electric field at a superficial portion of the body part point 1 in 320 changes in time at a different rate than in a deep location in the body part point 2 in graph 330.

Embodiments shown in FIGS. 2 and 3 can be combined or interleaved to achieve sophisticated control of neuronal excitation.

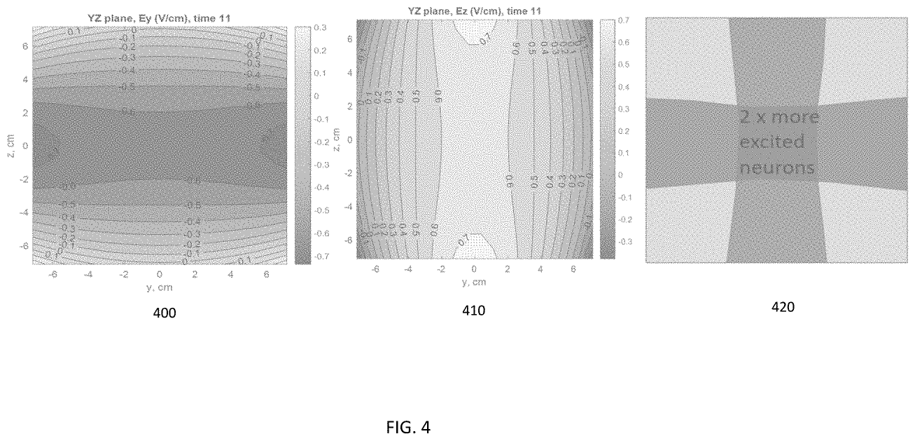

FIG. 4 illustrates how the regions of electrical field may be changed in time through changes in module activation so as to yield a smaller region of electrical change in tissue. Illustration 400 shows the electrical field caused by one configuration of activated modules. Soon thereafter, for example, within 50 microseconds, the configuration is changed to 410. The region of expected effect in tissue is shown in 420. FIG. 4 shows that by successively selecting different sets of modules for magnetization to yield different distributions 400 and 410 of high electric field, a smaller region 420 of nervous tissue can be excited. It is known that a rotating electric field is more efficient at stimulating neurons, as taught by Y. Roth et al in the article entitled “Rotational field TMS: Comparison with conventional TMS . . . ” published in Brain Stimulation 13 pages 900-907 (2020). Modules can be selected that achieve a rotational electric field as shown in FIGS. 3 and 4.

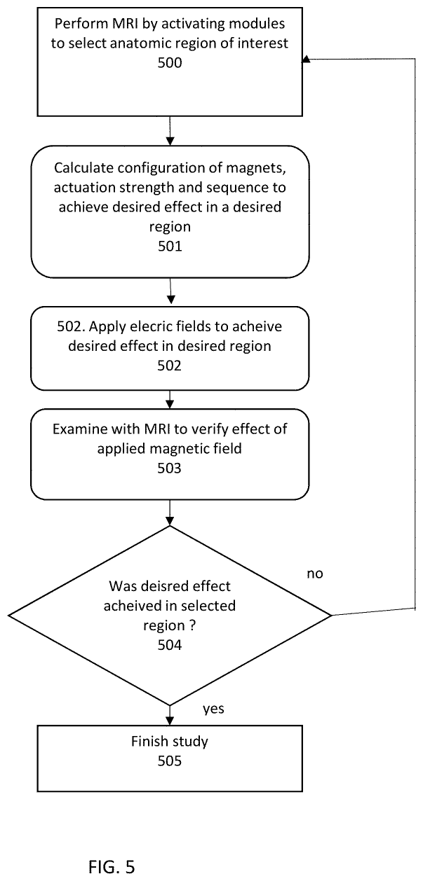

FIG. 5 demonstrates an embodiment of the method as applied to a patient or subject (human or otherwise). In operation 500, magnetic resonance images may be collected by using, for example, the modules in the assembly 120 in conjunction with radiofrequency antennas and/or gradient coils. The use of electropermanent magnets to collect images is described described in issued U.S. Pat. No. 10,908,240 and in related patents including US Pat. Pub. 20170227617, entitled “METHOD AND APPARATUS FOR MANIPULATING ELECTROPERMANENT MAGNETS FOR MAGNETIC RESONANCE IMAGING AND IMAGE GUIDED THERAPY, incorporated herein by reference.

The position of the body may be used to determine which magnetic modules to actuate, and with what degree of magnetization.

Which magnets and in what sequence the magnets should be activated to stimulate desired region is calculated at 501. Modules may be actuated to stimulate or modulate neurons in the selected region of interest at 502. An MRI may be used with appropriate pulse sequences to collect functional images at 503, for example, examining blood flow in the region. In operation 504, the images collected at 503 can be examined by a person or computer to verify that the application of electrical fields had the desired effect. If not, then the calculation at 500 may be repeated. It should be understood that many repetitions of various operation may be done until the end of the study 505.

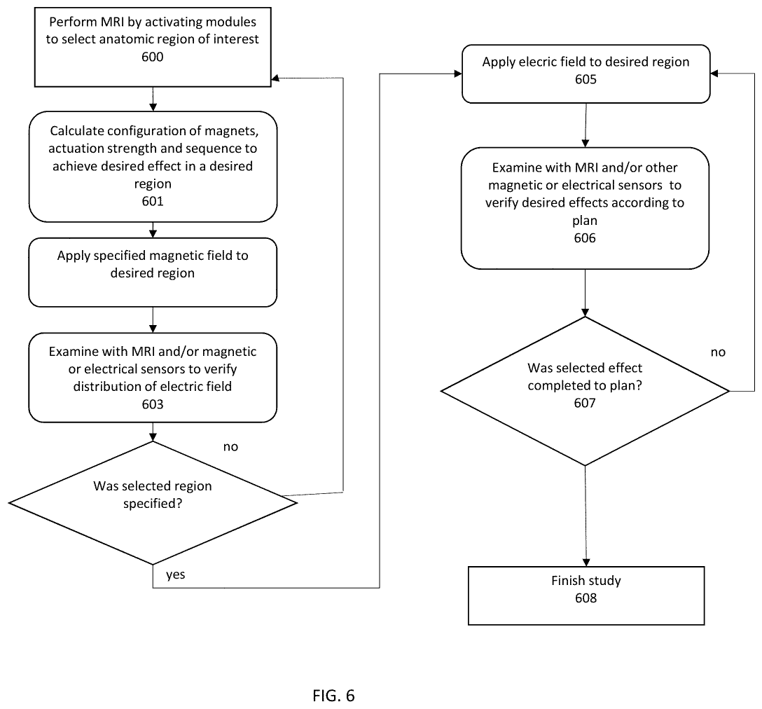

FIG. 6 illustrates an embodiment in which a magnetic resonance image is collected to verify correct region specification prior to application of modulation. An MRI is obtained by actuating magnetic modules to assist in localizing a region for modulation 600. A calculation is made (that may include data collected in operation 600) of the appropriate sequence of actuation of magnetic modules to modulate a region in a body part 601. A magnetic field is created in the body part 602 according to the calculation 601. An MRI pulse sequence is executed by actuating magnetic modules 603. The results of the MRI performed 603 are analyzed to verify that the correct region has been selected according to plan 604. If so, the next operation 605 may be executed. If not, some or all of operations 600-604 may be repeated. In operation 605, magnetic modules are actuated to apply an electric field to the selected region. An MRI pulse sequence may be executed to examine whether neuronal or nerve tissue in the selected region was modulated 606. This pulse sequence may use blood flow, blood volume, magnetocardiography, electrocardiography or other MRI techniques for the purpose of determining whether modulation has been according to a predetermined plan. The MRI of the prior operation is analyzed to seen if the plan has been executed correctly, and whether the planned number of repetitions has been executed 607. If so, the study is concluded. If not, some or all of operations 600-607 may be repeated. Disclosed embodiments may be controlled by, for example, a controller or control system 775 in wired or wireless communications with the assembly and having a processor 780 and power supply 785.

It should be understood that some of the operations in FIG. 5 or 6 may be omitted or be placed in different orders, and that sets of magnetic modules actuated in some operations may differ from the sets of magnetic modules actuated in other operations. It should be understood that the operations may be interrupted or otherwise changed according to the status of the animal subject (for example, in case of fibrillation). It should be understood that the subject may undergo multiple sessions of treatment with the operations of FIG. 5 or 6, for example, repeating the sessions weekly as needed to reduce symptoms of a disease.

It should be understood that the terms “actuation” or “firing” may represent partial or complete magnetization of one or more parts of one or more modules, with polarizations in one or more directions (e.g., South-North or North-South).

It should be understood that arcs or other patterns in the neuronal tissue may be created with the apparatus and method, which may be helpful in diagnosis or treatment or research into connected parts of the neuronal tissue.

It should be understood that the region of interest may be within the assembly (for example, the region may be in a hand) or may be near the assembly (for example, the region may be in the heart).

It should be understood that electric fields generated in deep or other selected structures with the invention may be used to cure or otherwise treat or diagnose diseases such as cancer.

It should be understood that the ability to generate electrical fields at various locations with different frequencies may be useful aside from stimulation, for example, to selectively actuate administered materials (for example, magnetoelectric particles).

It should be understood that since some of the magnetic modules may be placed close to the heart or tissue of interest, the electric fields may be very high (i.e., 700 volts/cm). Therefore, the electrical fields generated by the system may be sufficiently strong to cause electroporation or other tissue damage affecting conduction or conductivity for temporary effect, which may be useful in mapping or treatment planning, or permanent effect (which may be useful for therapy.

It should be understood that technical utility of the disclosed embodiments may include the ability to non-invasively modify cardiac conduction abnormalities or arrhythmias, which would be especially helpful when the abnormal foci causing such abnormalities are difficult or impossible to access with catheters.

Those skilled in the art will recognize, upon consideration of the above teachings, that the above exemplary embodiments and the control system may be based upon use of one or more programmed processors programmed with a suitable computer program. However, the disclosed embodiments could be implemented using hardware component equivalents such as special purpose hardware and/or dedicated processors. Similarly, general purpose computers, microprocessor based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out he above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as, for example, Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for selectively modulating neuronal tissue in a body comprising:
an assembly of a plurality of magnetic modules,
a controller to control the plurality of magnetic modules,
wherein at least one of the plurality of magnetic modules is controllable by the controller to be fired independently of another of the plurality of magnetic modules,
wherein the assembly is controllable by the controller to induce electric fields at different frequencies at different locations in a body part, the body part being near or partially or totally enclosed by the assembly, and
wherein the at least one of the plurality of magnetic modules is configured to collect an image of the body part.

2. The apparatus of claim 1, wherein the controller is configured to induce the electric fields at neural tissue in the body part at the neuronal tissue frequency-dependent threshold for stimulation so that deep sections of the body part may be stimulated or otherwise modulated to a different degree than other sections of the body part.

3. The apparatus of claim 1, wherein the at least one of the plurality of magnetic modules includes a magnetizable material and a coil of electrically conductive material.

4. The apparatus of claim 1, wherein the plurality of magnetic modules are positioned around the body part, and the body part is the brain, nerve root, heart, inner ear, or gut of an animal.

5. The apparatus of claim 1, wherein the electric fields induced in the body part are high enough to cause temporary or permanent changes in electrical conduction or conductivity in the body part.

6. The apparatus of claim 1, further comprising sensors configured to detect electrical or magnetic activity in the body part.

7. The apparatus of claim 1, wherein the controller is further configured to control successively actuate different combinations of the plurality of magnetic modules in the assembly to rotate the electric fields in space as a function of time.

8. The apparatus of claim 1, wherein the apparatus is further configured to implement anesthesia by applying the electric fields to nerves in or near teeth.

9. The apparatus of claim 1, wherein the image collection comprises magnetic resonance imaging, and wherein the apparatus is configured to collect the image before or after selective modulation of the body part to depict the localization of the electric fields.

10. A method of selectively modulating neuronal tissue in a body part, the method comprising:
providing an assembly of a plurality of magnetic modules and a controller to control the plurality of magnetic modules,
selectively actuating one or more magnetic modules in an assembly of a plurality of magnetic modules via the controller near to or enclosing the body part, wherein the selective actuation creates electric fields at different frequencies at different locations in the body part, and
collecting an image of the body part using at least one of the plurality of magnetic modules,
wherein the assembly is controllable by the controller to induce electric fields at different frequencies at different locations in a body part, the body part being near or partially or totally enclosed by the assembly.

11. The method of claim 10, wherein the controller is configured to induce the electric fields at neural tissue in the body part at the neuronal tissue frequency-dependent threshold for stimulation so that deep sections of the body part may be stimulated or otherwise modulated to a different degree than other sections of the body part.

12. The method of claim 10, wherein the selective actuation results in modulation of an arc or other pathway in the body part.

13. The method of claim 7, wherein the image collection comprises magnetic resonance imaging, and wherein the image is collected before or after selective modulation of the body part to depict the localization of the electric fields.

14. The method of claim 10, wherein the electric fields are applied to structures at selective locations in the body for diagnosis or treatment of cancers or other diseases.

15. The method of claim 10, wherein the electric fields are applied to administered materials at selective locations in the body, and wherein the administered materials are actuated for diagnosis or treatment of diseases.

16. The method of claim 10, wherein the electric fields are applied to electrically-sensitive tissues at selective locations in the body to temporarily alter properties of electrical conduction or conductivity.

17. The method of claim 10, wherein the electric fields are applied to electrically-sensitive tissues at selective locations in the body to temporarily alter properties of electrical conduction or conductivity.

18. The method of claim 10, further comprising detecting electric or magnetic fields originating in the body part via sensors that are located in or out of the modules and using the sensed electric or magnetic fields to bin or otherwise modify the image and/or the electrical fields created by the modules.

19. The method of claim 10, wherein anesthesia is implemented by applying the electric fields to nerves in or near teeth.

20. The method of claim 10, further comprising successively actuating different combinations of the magnetic modules in the assembly to rotate the electric fields in space as a function of time.
